(19) United States
(12) Patent Application Publication
(10) Pub. No.: US 2025/0312184 A1
(43) Pub. Date: Oct. 9, 2025

(54) SPLINT ARRANGEMENT

(71) Applicant: Colenski L.L.C., Beverly Hills, MI (US)

(72) Inventors: John GORSKI, Waterford, MI (US); Alexander COLEN, Beverly Hills, MI (US)

(73) Assignee: Colenski L.L.C., Beverly Hills, MI (US)

(21) Appl. No.: 19/098,238

(22) Filed: Apr. 2, 2025

Related U.S. Application Data
(60) Provisional application No. 63/575,943, filed on Apr. 8, 2024.

(51) Int. Cl. A61F 5/058

(52) U.S. Cl. CPC A61F 5/05875; A61F 5/05825

(57) ABSTRACT

Finger splint arrangements are disclosed herein. A finger splint arrangement may include a base section, a center section and a tip section that are connected together with wires such that base section, center section, and tip section are moveable independently from one another. Alternatively, a one-piece arrangement is disclosed that includes a ring member and a tip section defined by a pair of leg members that join to a tip member.

### CROSS REFERENCE TO RELATED APPLICATIONS

This application claims priority to U.S. Provisional Patent Application No. 63/575,943, filed on Apr. 8, 2024, the disclosure of which is incorporated herein by reference in its entirety.

### TECHNICAL FIELD

The present disclosure relates generally to a finger splint.

### BACKGROUND

A common injury that many people experience is a jammed finger or thumb tip. This may occur when a force is directed at a distal tip of the finger or thumb. Oftentimes, when the fingertip is jammed, the tendon can tear off the bone, i.e., an avulsion injury. In some instances, a small piece of the bone may also be separated with the tendon. Such instances are known as an avulsion fracture. Examples of the different injuries are illustrated in FIG. 1. These injuries are sometimes referred to as mallet finger.

In the past, treatment for such injuries involved surgery, which was both expensive and time consuming. More specifically, during surgery, the bone and/or tendon would be repaired, i.e., properly positioned, and the finger would be pinned straight to allow for healing of the tendon/bone.

Currently treatment methods have evolved to simply splinting the fingertip straight at the last joint for 6-8 weeks. There are a number of known splints for accomplishing this task. However, patient compliance is an issue that often adversely affects the outcome of patient treatment as the splint must be kept on the injured finger without removal for the entirety of the 6 to 8-week treatment period, 24 hours a day. Many patients remove the splits prematurely before the injury has fully healed for several reasons.

For example, known splints, such as one the one pictured in FIG. 2, often come with padding that gets wet and grimy, such that the patient simply removes the splint, or the splint simply falls off. Or the patient routinely removes the splint to wash their finger. Premature removal of the splint, however, may result in failed healing and/or deformity.

Further, trigger finger is a common condition that people experience. Trigger finger can be caused by a swelling of a tendon of a finger or a tendon tunnel through which the tendon runs, and can make it difficult to bend or straighten the finger.

### SUMMARY

Exemplary arrangements of finger splints are disclosed that can comfortably maintain a finger in a straightened position during healing from a finger or thumb injury, thereby increasing the likelihood of patient compliance during the healing process. Arrangements of the finger splints can also be used to apply pressure to a finger experiencing trigger finger, thereby relieving symptoms, and aiding in the healing process.

Finger splint arrangements for use in the treatment of finger injuries are disclosed herein. In one exemplary arrangement, a finger splint arrangement discloses a ring element sized to fit about a finger to be treated, a base section, a center section, a tip section, and at least a pair of wires.

The base member is fixedly connected to the ring element and the wires connect the tip section, center section, and base section such that the tip section, center section, and base section are moveable independently from one another.

In one exemplary arrangement, the base section includes a wall member, and a pair of leg members disposed on either side of the base member. Each leg member of the base section further comprises a channel that extends from a free end of the leg member, toward the wall member. Each channel may be configured to receive one wire of the at least a pair of wires.

The wires may be constructed of a malleable material. With this arrangement, selective positioning of the different sections of the finger splint arrangement is achievable.

In one exemplary arrangement, the center section is defined by first and second ends and a pair of channels that extend between the first and second ends of the center section. The wires extend through these channels to connect the center section to the base section. In one exemplary arrangement, the center section has a generally uniform length.

In one exemplary arrangement, the tip member is defined by leg members that extend from a first end and arc toward a common distal tip member. The tip member is defined by a length and width that is configured to overlay a fingernail such that the tip member may be adhered to the fingernail to secure the position of the finger splint arrangement.

In one exemplary arrangement, the tip member is defined by leg members that extend from a first end and arc toward a common distal tip member. Each leg member of the tip member includes a channel extending from the first end toward the tip member and includes a channel extending from the first end toward the tip member. The wires extend into these channels to secure the center section to the tip section.

In one exemplary arrangement, each of the base section, center section and tip section are configured with a curve when viewed in profile.

In one exemplary arrangement, each of the base section, center section, and tip section are constructed of a formable material.

In one exemplary arrangement, the ring element includes a groove formed in a bottom surface thereof. In addition, an area of reduced thickness may be provided along an inside surface of the ring element, in the same area of the ring element where the groove is formed to facilitate removal of the ring element, if needed. The ring element may also include one or more cutouts extending through the thickness of the ring element to allow for airflow, as well as reduced weight.

In one exemplary arrangement, a first pair of spacer elements are disposed between the tip member and the center section. A second pair of spacer elements are disposed between the center section and the base member. The spacer elements may be constructed of a different material than the base member, center section, and the tip member. For example, the material of the spacer element may have a lower durometer than the remainder of the finger splint arrangement.

In one exemplary arrangement, a one-piece finger splint arrangement is provided. This arrangement comprises a ring element and an extended tip section. The tip section comprises a pair of leg members and a tip member, wherein an open area is disposed between the ring element, the leg members, and the tip member.

In one exemplary arrangement, the tip section is integrally formed with the ring element. A base member may also be provided, with the base member being attached to the ring element and the leg members extending from the base member. The leg members arc toward the tip member.

In one exemplary arrangement, the ring element and extended tip section are constructed of a self-molding thermoplastic material that can be molded by application of a small amount of heat. The application of a small amount of heat can include immersing the finger splint arrangement including the ring element and extended tip section in hot water.

In one exemplary arrangement, the ring element includes a groove formed in a bottom surface. The ring element can further include an area of reduced thickness along an inside surface of the ring element in a same area of the ring element where the groove is formed. On a front face of the base member, an indicium may be provided thereon that is indicative of the ring finger size of the finger splint arrangement. The indicium may be formed integrally therein, for example through molding.

In an exemplary arrangement, the finger splint is configured to be worn either with the extended tip section extending over a top of a finger to which the finger splint arrangement is attached, or with the extended tip section extending away from the finger to which the finger splint arrangement is attached, toward a hand and under an area of a palm of the hand.

### BRIEF DESCRIPTION OF THE DRAWINGS

The foregoing and other features and advantages of the present disclosure will become apparent to those skilled in the art to which the present disclosure relates upon reading the following description with reference to the accompanying drawings, in which:

FIGS. 1A-1C are cross-sectional illustrations of representative fingertip injuries;

FIG. 2 is an example of a prior art splint;

FIG. 3 is an exploded view of a splint for a fingertip according to an exemplary arrangement;

FIG. 4 is a perspective view of the splint of FIG. 3; in an assembled configuration;

FIG. 5 is a side elevational view of the splint of FIG. 4;

FIG. 6 is a top plan view of the splint of FIG. 4;

FIG. 7 is a front elevational view of a splint according to another exemplary arrangement; and

FIG. 8 is a perspective view of the split of FIG. 7.

FIG. 9 is an exploded view of a splint according to another exemplary arrangement;

FIG. 10 is a perspective view of a splint of FIG. 9, in an assembled configuration;

FIG. 11 is a side elevational view of the splint of FIG. 10;

FIG. 12 is front elevational view of the splint of FIG. 10;

FIG. 13 is an enlarged view of the encircled area 13 in FIG. 10;

FIG. 14 is a bottom plan view of the splint of FIG. 10;

FIG. 15 is a perspective view of a splint according to another exemplary arrangement;

FIG. 16 is a side elevational view of the splint of FIG. 15;

FIG. 17 is a side elevational view of the splint of FIG. 15, taken from an opposite side as FIG. 16;

FIG. 18 is an elevational view showing an inside surface of the splint of FIG. 15;

FIG. 19 is a bottom plan view of the splint of FIG. 15; and

FIG. 20 a front elevational view of the splint of FIG. 15.

FIG. 21 shows an example splint worn to treat mallet finger.

FIGS. 22A-22B show an example splint worn to treat trigger finger.

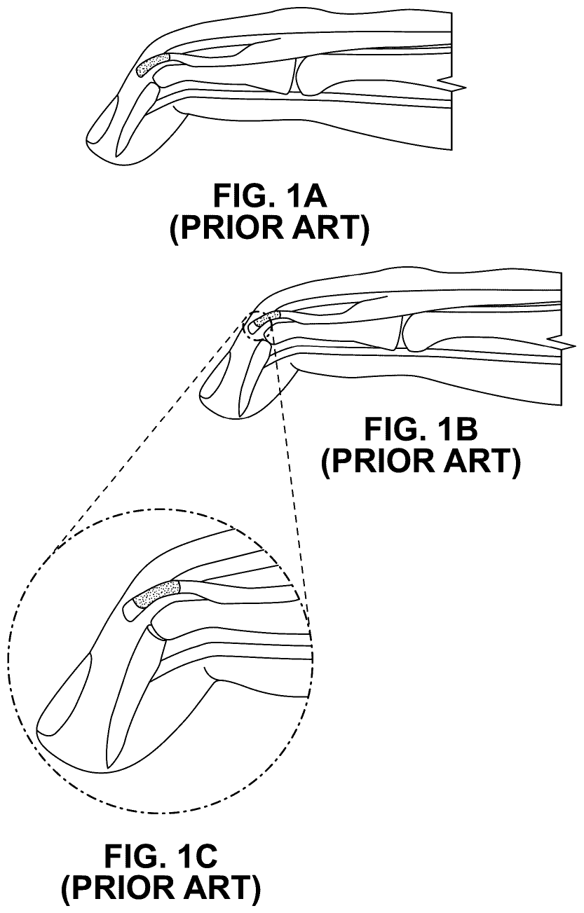

### DETAILED DESCRIPTION

Referring now to the discussion that follows, and to the drawings, illustrative approaches to the disclosed systems and methods are shown in detail. Although the drawings represent some possible approaches, the drawings are not necessarily to scale and certain features may be exaggerated, removed, or partially sectioned to better illustrate and explain the present disclosure. Further, the descriptions set forth herein are not intended to be exhaustive or otherwise limit or restrict the claims to the precise forms and configurations shown in the drawings and disclosed in the following detailed description.

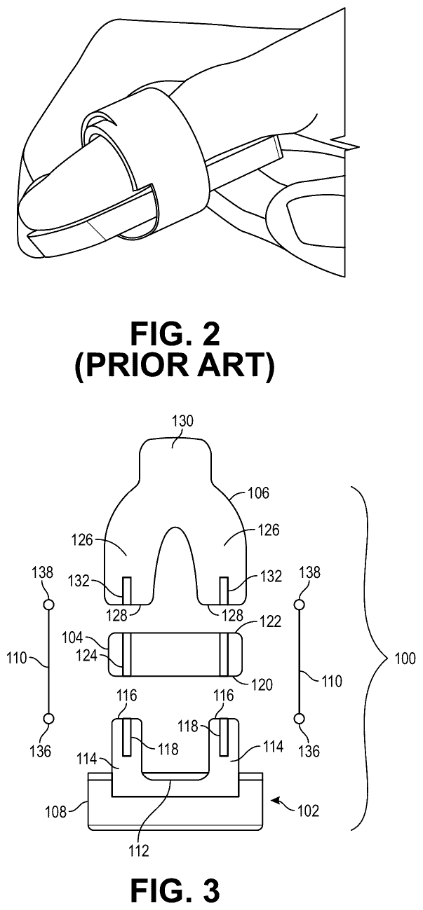

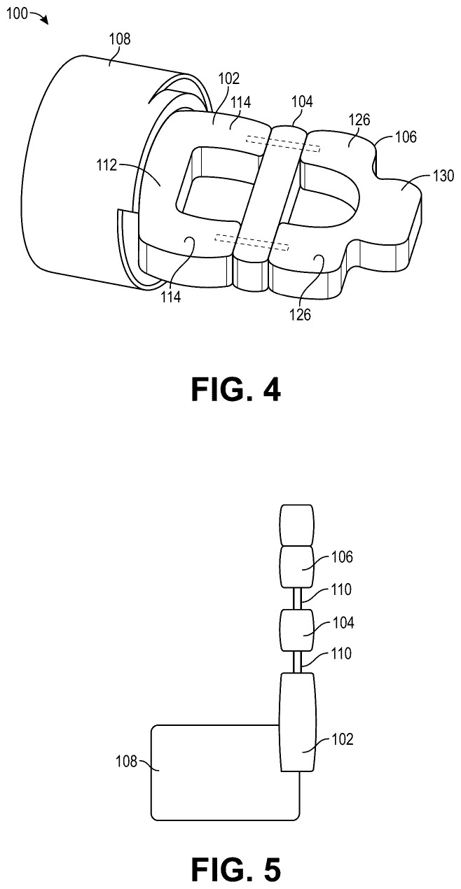

Referring to FIGS. 3-6, an exemplary arrangement of a finger splint arrangement 100 is illustrated. The finger splint arrangement 100 includes a base section 102, a center section 104, and a tip section 106. The base section 102 is fixed to a ring element 108. Wires 110 connect the tip section 106, center section 104, and base section 102 together.

In one exemplary arrangement, the ring element 108 is continuous and uninterrupted. The ring element 108 is configured to fit around a finger at a location proximal to the distal interphalangeal joint.

The base section 102 is defined by a wall member 112 and a pair of leg members 114 that are spaced apart from each other. The leg members 114 are arranged to be parallel to one another. Each leg member 114 is defined by an end 116. A channel 118 extends inwardly from the end 116 in each leg member 114, toward the ring element 108.

In one exemplary arrangement, the wall member 112 is integrally formed with the leg members 114. The wall member 112 may have a length that is shorter than a length of the leg members 114 so as to form an open area between the leg members 114 and the wall member 112.

The center section 104 is defined by first and second ends 120 and 122. In one exemplary arrangement, the first and second ends 120 and 122 are generally parallel with each other such that the center section 104 has a generally uniform length. Disposed within the center section 104 are two parallel channels 124. Channels 124 extend from the first end 120 through the second end 122, such that the channels 124 extend the entire length of the center section. Channels 124 are configured to be generally aligned with channels 118 in the base section 102, as will be explained in further detail below.

The tip section 106 is defined by leg members 126 that extend from a first end 128. The leg members 126 arc toward a common distal tip member 130. The tip member 130 is centered between the leg members 126. The tip member 130 is configured to attach directly to a fingernail with adhesive. In this way, there is no need for any cumbersome straps or adhesive tapes. This arrangement ensures stability without impeding natural nail growth or causing discomfort.

Extending toward the tip member 130 from the first end 128 of each leg member 126 is a channel 132. The channel 132 terminates within each leg member 126. Channels 132 are configured to be generally aligned with channels 124 in the center section 104, as will be explained in further detail below.

As shown in FIG. 3, the finger splint arrangement 100 further includes a pair of malleable wires 110. The wires 110 are initially positioned within the channels 118, with a first end 136 fixed therewithin. A second end 138 of each wire 110 is then threaded through each channel 124 of the center section 104, until it exits the channels 124 of the center section 104. The second end 138 of each wire 110 is then positioned within each channel 132 of the tip section 106 and fixed within the channel 132. Once the wires 110 are positioned in this manner, the base section 102, the center section 104, and the tip section 106 are all connected, but each section may move independently. In addition, the angle of extension of a fingertip when placed in the splint arrangement 100 may be selectively adjusted due to the independent movement of the differing sections, prior to the tip member 130 being secured to the fingernail. With this configuration, the therapist or clinician can set an optimal angle for the fingertip and subsequent healing.

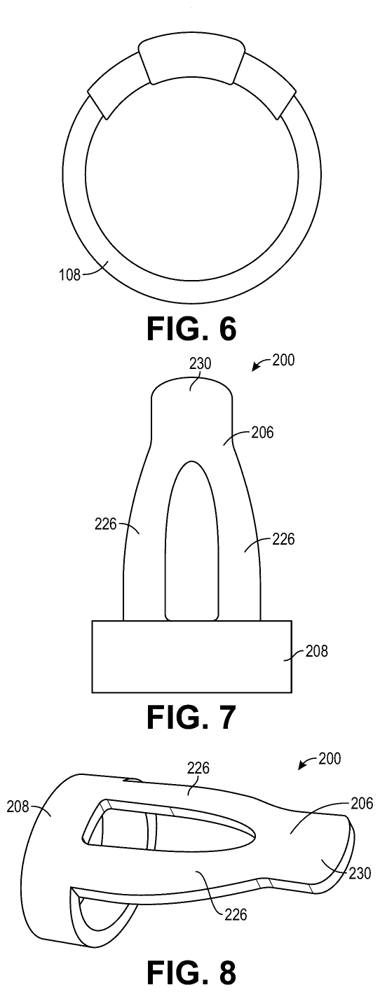

The finger splint arrangement 100 is constructed of a lightweight and durable material, and each section 102, 104 and 106 is slightly curved, as shown in FIG. 6, such that the finger splint arrangement 100 conforms to the natural curvature of the finger and nail. In one exemplary arrangement, the base section 102, center section 104, and tip section 106 are constructed of a polymer material. Alternatively, these sections 102, 104, and 106 may be constructed of other materials, such as metal, composite, or self-molding thermoplastic material which may be molded with a small amount of heat and/or hot water applied.

Overall, the finger splint arrangement 100 offers an innovative solution to address the limitations of conventional splinting methods, promising improved patient comfort, compliance, and ultimately, better treatment outcomes for mallet finger injuries.

An alternative exemplary arrangement of a finger splint arrangement 200 is shown in FIGS. 7-8. Finger splint arrangement 200 includes a ring element 208 and an extended tip member section 206. The tip member section 206 is attached to the ring element 208 and may be integrally formed with the ring element 208. The tip member section 206 includes a pair of leg members 226.

The leg members 226 arc toward a common distal tip member 230, leaving an open area therebetween. The tip member 230 is centered between the leg members 226. The tip member 230 is configured to attach directly to a fingernail with adhesive, again avoiding the need for any cumbersome straps or adhesive tapes. Similar to the splint arrangement 100, splint arrangement 200 may also be constructed of a polymer material, a metal, composite material. The material may further be self-molding thermoplastic material that can be molded with the application of a small amount of heat, for example by immersing the finger splint arrangement 200 in hot water. The two leg members 226 provide for a stable fixation, while still allowing flexibility of the finger splint arrangement 200.

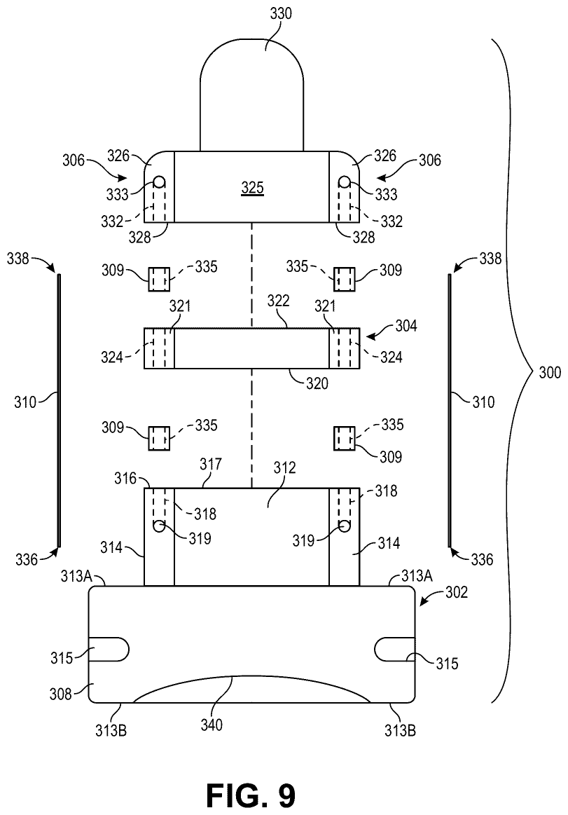

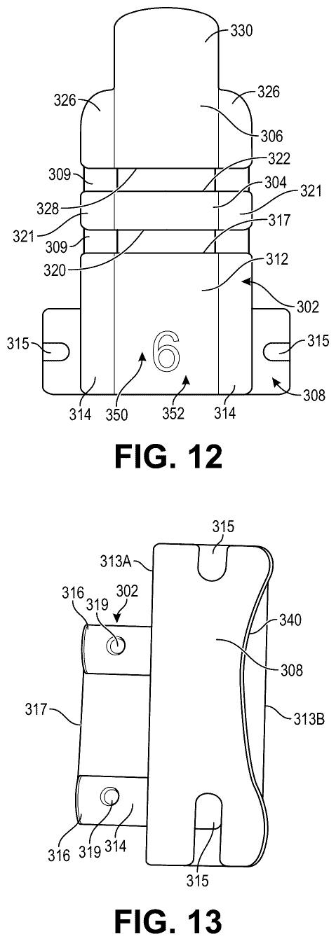

Referring to FIGS. 9-14, an exemplary arrangement of a finger splint arrangement 300 is illustrated. The finger splint arrangement 300 is similar to the exemplary arrangement shown in FIGS. 3-6. More specifically, the finger splint arrangement 300 includes a base section 302, a center section 304, and a tip section 306. The base section 302 is fixed to a ring element 308. Wires 310 (best seen in FIG. 9) connect the tip section 306, center section 304, and base section 302 together. Two sets of spacer elements 309 are provided. A first set of spacer elements 309 are positioned between the tip section 306 and the center section 304 and a second set of spacer elements 309 are positioned between the center section 304 and the base section 302. The wires 310 are threaded through a portion of the base section 302, spacer elements 309, center section 304 and into the tip section 306, as will be explained in further detail below.

In one exemplary arrangement, the ring element 308 is continuous about a circumference. The ring element 308 is configured to fit around a finger at a location proximal to the distal interphalangeal joint. In one exemplary arrangement, cutouts 315 may be provided through the ring element 308, the cutouts 315 being spaced from top and bottom ends 313A, 313B, respectively, of the ring element 308. The cutouts 315 serve to make the finger splint arrangement 300 lighter, as well as more flexible, when worn. In addition, the cutouts 315 allow airflow to the finger when worn.

The base section 302 is defined by a wall member 312 and a pair of leg members 314 that are spaced apart from each other. The leg members 314 are arranged to be parallel to one another. In one exemplary arrangement, each leg member 314 has an end 316 that terminates at a top edge 317 of the wall member 312. Each leg member 314 has a thickness that is greater than the thickness of the wall member 312. In one exemplary arrangement, the leg members 314 have a circular cross-section. A channel 318 extends inwardly from the end 316 toward the ring element 308. The channel 318 terminates in an opening 319 extending through an inside surface of the leg members 314, as best seen in FIG. 9.

In one exemplary arrangement, the wall member 312 is integrally formed with the leg members 314. In addition, in one exemplary arrangement, the wall member 312 may be integrally formed with the ring element 308.

The center section 304 is defined by first and second ends 320 and 322. In one exemplary arrangement, the first and second ends 320 and 322 are generally parallel to each other such that the center section 304 has a generally uniform length. Disposed within the center section 304 are two parallel leg members 321, similar in shape to the leg members 314 of the base section 302. Disposed within the leg members 321 are channels 324. The channels 324 extend from the first end 320 through the second end 322, such that the channels extend the entire length of the center section 304. The channels 324 are configured to be generally aligned with the channels 318 of the leg members 314 in the base section 302, as will be explained in further detail below. Similar to the leg members 314 in the base section 302, leg members 321 have a thickness that is greater than the thickness of the wall member 312. The leg members 321 may also be formed with a circular cross-section.

The tip section 306 is defined by leg members 326 one on either end with a center element 325 therebetween. The leg members 326 extend from a first end 328 of the tip section 306. A distal tip member 330 extends from the center element 325 of the tip section 306. The tip member 330 is centered between the leg members 326. The tip member 330 is configured to attach directly to a fingernail with adhesive. In this way, there is no need for any cumbersome straps or adhesive tapes. This arrangement further ensures stability without impeding natural nail growth or causing discomfort.

Extending toward the tip member 330 from the first end 328 of each leg member 326 is a channel 332. The channel 332 terminates within each leg member 326 at an opening 333, as best seen in FIG. 9. Channels 332 are configured to be generally aligned with channels 324 in the center section 304, as will be explained in further detail below.

As stated above, the first pair of spacer elements 309 are positioned between the tip section 306 and the center section 304. The second pair of spacer elements 309 are positioned between the center section 304 and the base section 302. Each of the spacer elements 309 includes a channel 335. The channels 335 extend through the length of the spacer, as shown in FIG. 9. In one exemplary arrangement, the spacer elements 309 have a width that that slightly smaller than the width of the leg members 314, 321, and 326. In one exemplary arrangement, the spacer elements 309 have a circular cross-section. The width of all of the leg members 314, 321, and 326 may be the same.

In one exemplary arrangement, the material of the spacer elements 309 is more compliant than the material of the remainder of the finger splint arrangement 300 (i.e., the base section 302, the center section 304, and the tip section 306). For example, the material of the spacer elements 309 may be constructed of a material having a lower durometer than that of the base section 302, center section 304 and the tip section 306.

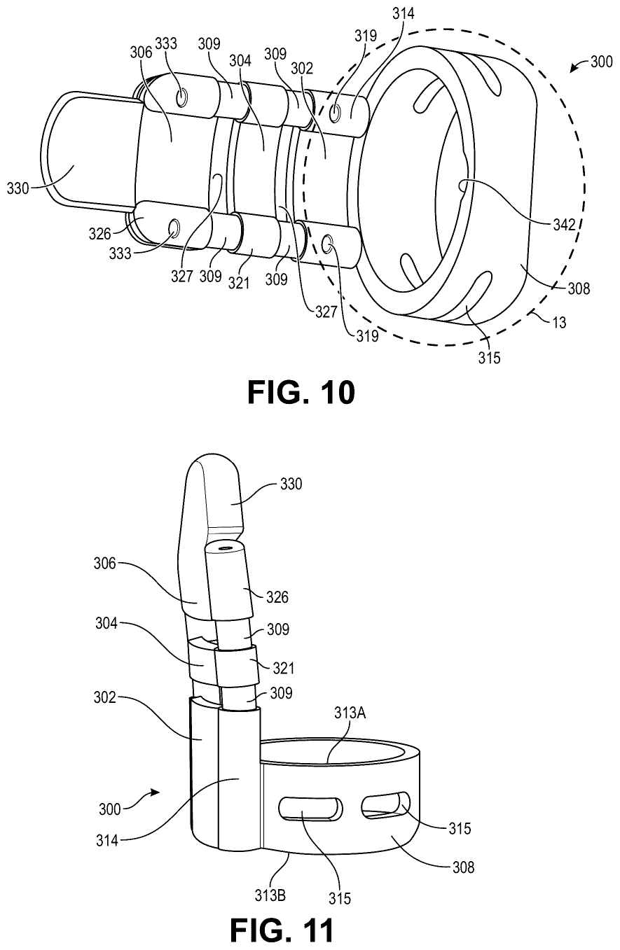

Similar to the arrangement 100 which is shown in FIG. 3, the finger splint arrangement 300 further includes a pair of malleable wires 310. The wires 310 are positioned within the channels 318, through openings 319, with a first end 336 fixed within the leg members 314. A second end 338 of each wire 310 is then threaded through each channel 355 of the first pair of spacer elements 309, the channels 324 of the center section 304, and through each channel 335 of the second pair of spacer elements 309. After exiting the channels 335 of the second pair of spacer elements 309, the second end 338 of each wire is then positioned within the channels 332 of each leg member 326. The second end 338 may be pulled through the opening 333 to bring the spacer elements 309 into frictional engagement with base section 302, the center section 304, and the tip section 306. Once arranged in this manner, the second end 338 may be fixed within the leg member 326. Once the wires 310 are positioned in this manner, the base section 302, the center section 304, and the tip section 306 are all connected, with cutout or void sections 327 (see FIG. 10) between each section, but each of the base section 302, center section 304 and tip section 306 may move independently. Due to the material of the spacer elements 309, the spacer elements 309 may act as hinge elements to allow for the articulation of the finger splint arrangement 300. For example, the angle of extension of a fingertip when placed in the splint arrangement 300 may be selectively adjusted due to the independent movement of the different sections, prior to the tip member 330 being secured to the fingernail. With this configuration, the therapist or clinician can set an optimal angle for the fingertip and subsequent healing.

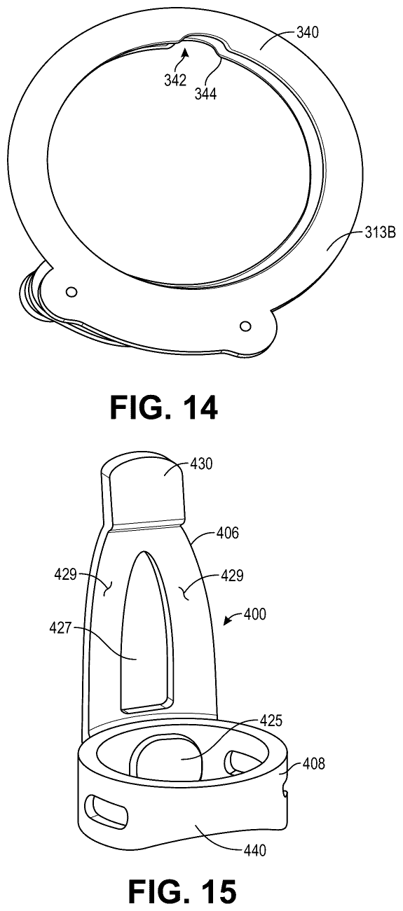

The finger splint arrangement 300 is constructed of a lightweight and durable material, and each section 302, 304 and 306 is slightly curved, as shown in FIG. 14, such that the finger splint arrangement 300 conforms to the natural curvature of the finger and nail. In one exemplary arrangement, the base section 302, center section 304, and tip section 306 are constructed of a polymer material. Alternatively, these sections 302, 304, and 306 may be constructed of other materials, such as metal, composite, or thermoplastic (self-molding) material which may be molded with a small amount of heat and/or hot water applied. For example, use of a thermoplastic material would enable a user to heat in warm water to shape the finger splint arrangement 300. Once cooled, the material will retain the unique shape. The spacer elements 309 may be constructed of [insert material example] that allow for compressibility of the spacer elements 309.

In another exemplary arrangement, referring to FIG. 13, the ring element 308 is provided with a grooved surface 340 in the bottom end 313B. The grooved surface 340 provides improved flexing of a finger over designs having the ring element 308 being of uniform width.

In one exemplary arrangement, the ring element 308 may also have an area 342 of reduced thickness. More specifically, referring to FIG. 14, the thickness of the ring element 308 is reduced along an inside surface 344 of the ring element 308, in the same area where the grooved surface 340 is formed. With this arrangement, should there be a need to cut off the finger splint arrangement 300 for any reason, such as excess swelling, the area 342 of reduced thickness facilitates getting scissors between the finger and the ring element 308.

As the finger splint arrangement 300 is configured to fit snugly on a finger, in one exemplary arrangement, the finger splint arrangements 300 are configured in different sizes that generally follow traditional ring sizes. To aid in distinguishing between the different sizes, in one exemplary arrangement, the size may be provided on a front surface 350 of the base section 302. More specifically, an indicium 352 that is indicative of the size of the finger splint arrangement 300 may be molded or otherwise imprinted into the base section 302.

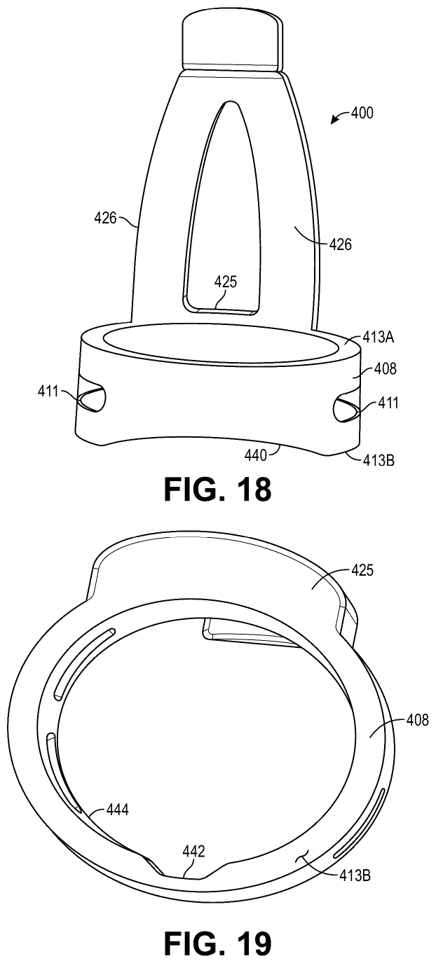

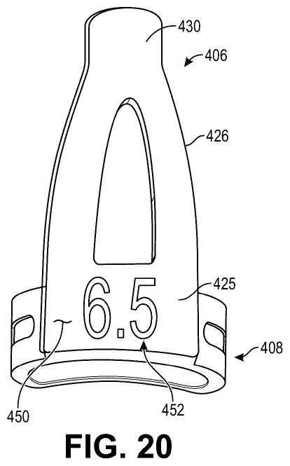

A further alternative exemplary arrangement of a finger splint arrangement 400 is shown in FIGS. 15-20. Finger splint arrangement 400 is similar to the finger splint arrangement 200. Finger splint arrangement 400 includes a ring element 408 and an extended tip member section 406. The tip member section 406 is attached directly to the ring element 408 and may be integrally formed with the ring element 408. The tip member section 406 includes a pair of leg members 426 connected to a base element 425 and a distal tip member 430.

The leg members 426 are both connected to the base element 425 at one end, and arc toward the common distal tip member 430, leaving an open area 427 therebetween. The open area 427 allows airflow to the finger when worn, as well as making the finger splint arrangement 400 lightweight. The two leg members 426 provide for a stable fixation, but still allowing flexibility of the finger splint arrangement 400.

The tip member 430 is centered between the leg members 426 such that second ends of each leg member 426 converge toward a central axis through the tip member section 406. The tip member 430 is configured to attach directly to a fingernail with adhesive, again avoiding the need for any cumbersome straps or adhesive tapes. To facilitate attachment, the tip member 430 may be constructed with a greater thickness than the leg members 426 such that the tip member 430 extends outwardly from an inside surface 429.

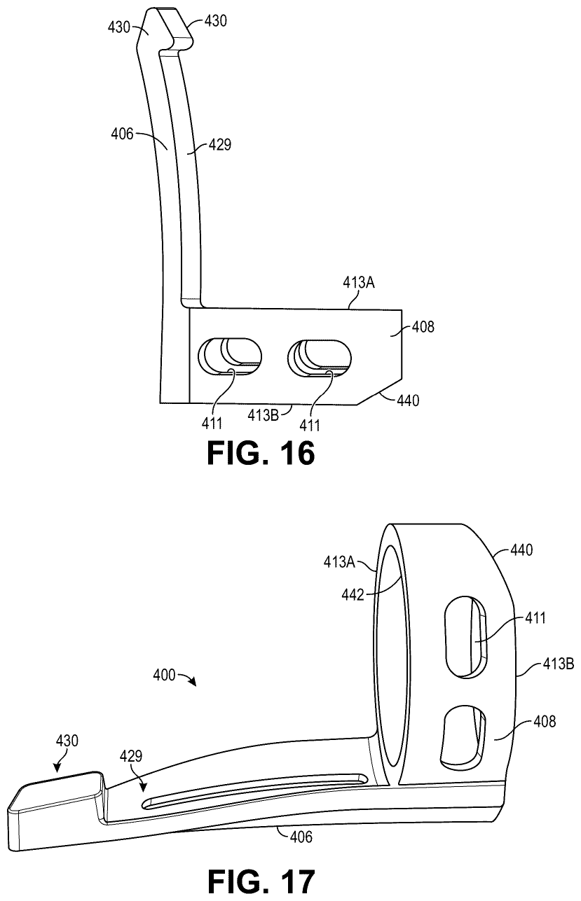

In one exemplary arrangement, the ring element 408 is continuous about a circumference. The ring element 408 is configured to fit around a finger at a location proximal to the distal interphalangeal joint. In one exemplary arrangement, cutouts 411 may be provided through the ring element 408, similar to the arrangement 300. For example, referring to FIG. 16, the cutouts 411 may be spaced from top and bottom ends 413A, 413B, respectively, of the ring element 408. The cutouts 411 serve to make the finger splint arrangement 400 lighter, as well as more flexible, when worn. In addition, the cutouts 411 allow airflow to the finger when worn.

Similar to the arrangement 300, the ring element 408 is provided with a grooved surface 440 in the bottom end 413B. The grooved surface 440 provides improved flexing of a finger over designs having the ring element 408 being of uniform width about its circumference.

In one exemplary arrangement, the ring element 408 may also have an area 442 of reduced thickness. More specifically, as best seen in FIG. 19, the thickness of the ring element 408 is reduced along an inside surface 444 of the ring element 404, in the same area where the grooved surface 440 is formed. With this arrangement, should there be a need to cut off the finger splint arrangement 400 for any reason, such as excess swelling, the area 442 of reduced thickness facilitates getting scissors between the finger and the ring element 408.

As the finger splint arrangement 400 is configured to fit snugly on a finger, in one exemplary arrangement, the finger splint arrangement 400 can be configured in different sizes that generally follow traditional ring sizes. To aid in distinguishing between the different sizes, the size may be provided on a front surface 450 of the base element 425. More specifically, an indicium 452 that is indicative of the size of the finger splint arrangement 400 may be molded or otherwise imprinted into the base element 425.

Similar to the arrangements 100, 200 and 300 disclosed herein, splint arrangement 400 may also be constructed of a lightweight and durable material, such as a polymer or thermoplastic material. In an example, the splint arrangement 400 is constructed as a single unit of a self-molding thermoplastic material. The thermoplastic material may be molded by application of a low amount of heat, for example by immersing the splint arrangement 400 in hot water. Other suitable materials may also be used. Further, the tip member section 406 is slightly curved in profile, as shown in FIGS. 16-17, such that the finger splint arrangement 400 conforms to the natural curvature of the finger and nail.

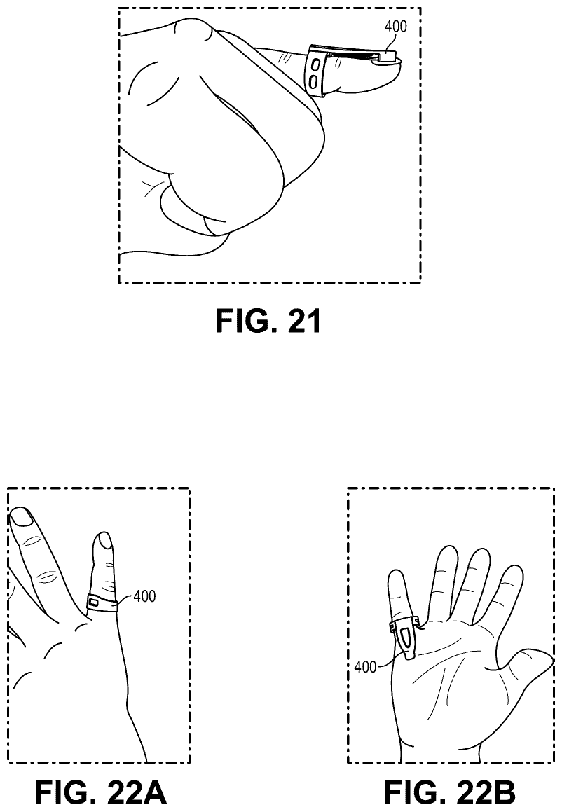

FIG. 21 shows an example of a patient wearing the splint arrangement 400 to treat mallet finger. As can be seen, the tip section 406 extends over the top of a finger of the patient. The tip member 430 is adhered to a finger nail of the patient.

In addition to treatment of mallet finger, the splint finger arrangements 200, 400 can be used for the treatment of trigger finger. Trigger finger is a condition where, due to swelling of a tendon in a finger or a surrounding tunnel through which the tendon passes, a person cannot bend or straighten a finger, or can do so only with difficulty. The finger splint arrangements 200, 400, can be used to treat this condition by placing the splint on the affected finger with the tip section extending away from the finger, under the palm of the patient. Worn in this way, the finger splint arrangements 200, 400 can apply pressure on the area of swelling of the tendon or tendon tunnel, thereby relieving the symptoms and aiding the healing process.

FIGS. 22A-22B show two views of a patient wearing the finger splint arrangement 400 for treating trigger finger. The tip section extends toward the hand, and underneath the palm.

Other arrangements are possible within the scope of the disclosure. For example, an arrangement like finger splint arrangement 100 or finger splint arrangement 300, without the center section could be imagined. This arrangement would include a base section and tip section, the base section and tip section being connected by two or more wires, in a manner similar to the arrangement 100. As another example, an arrangement like finger splint arrangement 200 or finger splint arrangement 400 may include wires, for example, in the leg sections, or a single wire running from the base and the tip, arranged between the leg members.

What have been described above are examples of the present disclosure. It is, of course, not possible to describe every conceivable combination of components or methodologies for purposes of describing the present invention, but one of ordinary skill in the art will recognize that many further combinations and permutations of the present invention are possible. While certain novel features of this disclosure shown and described below are pointed out in the annexed claims, the disclosure is not intended to be limited to the details specified, since a person of ordinary skill in the relevant art will understand that various omissions, modifications, substitutions and changes in the forms and details of the disclosure illustrated and in its operation may be made without departing in any way from the spirit of the present disclosure. Accordingly, the present disclosure is intended to embrace all such alterations, modifications, and variations that fall within the scope of the appended claims. As used herein, the term “includes” means includes but not limited to, the term “including” means including but not limited to. The term “based on” means based at least in part on. Additionally, where the disclosure or claims recite “a,” “an,” “a first,” or “another” element, or the equivalent thereof, it should be interpreted to include one or more than one such element, neither requiring nor excluding two or more such elements. No feature of the disclosure is critical or essential unless it is expressly stated as being “critical” or “essential.”

What is claimed is:

1. A finger splint arrangement for use in treatment of finger injuries, the arrangement comprising:
a ring element sized to fit about a finger to be treated;
a base section;
a center section;
a tip section; and
at least one pair of wires;
wherein the base section is fixedly connected to the ring element; and
wherein the at least one pair of wires connects the tip section, the center section, and the base section such that the tip section, the center section, and the base section are able to move independently from one another.

2. The finger splint arrangement of claim 1, wherein the base section includes a wall member, and a pair of leg members disposed on either side of the base section.

3. The finger splint arrangement of claim 2, wherein each leg member of the pair of leg members of the base section comprises first and second leg ends, wherein a channel extends at least partially through each leg member of the pair of leg members of the base section, the channel being configured to receive one of the at least one pair of wires.

4. The finger splint arrangement of claim 1, wherein the at least one pair of wires are constructed of a malleable material.

5. The finger splint arrangement of claim 3, wherein the center section is defined by first and second ends and a pair of channels extends between the first and second ends of the center section, the pair of channels of the center section generally aligning with the channel of each leg member of the pair of leg members of the base section and wherein one wire of the at least one pair of wires extends through each channel of the pair of channels of the center section.

6. The finger splint arrangement of claim 5, wherein the first and second ends of the center section are generally parallel to each other such that the center section has a generally uniform length.

7. The finger splint arrangement of claim 1, wherein the tip section is defined by leg members that extend from a first end and arc toward a common distal tip member.

8. The finger splint arrangement of claim 1, wherein the tip section includes a distal tip member defined by a length and a width that is configured to overlay a fingernail such that the distal tip member is attachable to the fingernail with adhesive.

9. The finger splint arrangement of claim 5, wherein the tip section includes first and second leg members disposed on either side of the tip section, and a distal tip member, and wherein each of the leg members of the tip section includes a channel extending from a first end toward the distal tip member; the channel of each of the leg members generally aligning with the pair of channels of the center section and wherein one wire of the at least one pair of wires extends into the channel of each of the leg members of the tip section.

10. The finger splint arrangement of claim 1, wherein each of the base section, the center section, and the tip section are curved when in an assembled configuration and viewed in profile.

11. The finger splint arrangement of claim 1, wherein the ring element includes a groove formed in a bottom surface.

12. The finger splint arrangement of claim 11, wherein the ring element includes an area of reduced thickness along an inside surface of the ring element in a same area of the ring element where the groove is formed.

13. The finger splint arrangement of claim 1, wherein the ring element includes one or more cutouts extending through a thickness of the ring element.

14. The finger splint arrangement of claim 1, further comprising a first pair of spacer elements disposed between the tip section and the center section, and a second pair of spacer element disposed between the center section and the base section.

15. A finger splint arrangement, comprising:
a ring element; and
an extended tip section; wherein the extended tip section comprises a pair of leg members and a tip member; wherein an open area is disposed between the ring element, leg members, and the tip member.

16. The finger splint arrangement of claim 15, wherein the extended tip section is integrally formed with the ring element.

17. The finger splint arrangement of claim 16, wherein the ring element and extended tip section are constructed of a self-molding thermoplastic material that can be molded by application of a small amount of heat.

18. The finger splint arrangement of claim 17, wherein the application of a small amount of heat can include immersing the finger splint arrangement including the ring element and extended tip section in hot water.

19. The finger splint arrangement of claim 15, wherein the leg members arc toward the tip member.

20. The finger splint arrangement of claim 15, further comprising a base member attached to the ring element, wherein the leg members extend from the base member.

21. The finger splint arrangement of claim 15, wherein the tip member extends outwardly from an inside surface of the leg members.

22. The finger splint arrangement of claim 15, wherein the ring element includes a groove formed in a bottom surface.

23. The finger splint arrangement of claim 22, wherein the ring element includes an area of reduced thickness along an inside surface of the ring element in a same area of the ring element where the groove is formed.

24. The finger splint arrangement of claim 15, wherein the finger splint arrangement is configured to be worn either with the extended tip section extending over a top of a finger to which the finger splint arrangement is attached, or with the extended tip section extending away from the finger to which the finger splint arrangement is attached, toward a hand and under an area of a palm of the hand.
